(19) United States
(12) Patent Application Publication
(10) Pub. No.: US 2013/0304102 A1
(43) Pub. Date: Nov. 14, 2013

(54) METHODS AND APPARATUS FOR STIMULATING STRETCH RECEPTORS IN THE VASCULATURE

(71) Applicants: Yossi Gross, Moshav Mazor (IL); Chung Hao Yeh, San Mateo, CA (US); Christopher Ken, San Mateo, CA (US); Brent Seybold, Santa Clara, CA (US); Jennifer Gong, San Jose, CA (US)

(72) Inventors: Yossi Gross, Moshav Mazor (IL); Chung Hao Yeh, San Mateo, CA (US); Christopher Ken, San Mateo, CA (US); Brent Seybold, Santa Clara, CA (US); Jennifer Gong, San Jose, CA (US)

(73) Assignee: Vascular Dynamics, Inc., Mountain View, CA (US)

(21) Appl. No.: 13/889,643

(22) Filed: May 8, 2013

Related U.S. Application Data
(60) Provisional application No. 61/644,911, filed on May 9, 2012.

(51) Int. Cl. A61B 17/12

(52) U.S. Cl. CPC A61B 17/12
USPC 606/158

(57) ABSTRACT

Stimulation of a stretch receptor response to the cardiac cycle is enhanced by constraining a region of a blood vessel wall upstream and/or downstream of a baroreceptor. The constraint may be external or internal and will typically be effected using a circumferential restraint placed over or within the blood vessel.

### CROSS-REFERENCE TO RELATED APPLICATIONS

This application claims the benefit of U.S. Provisional Application No. 61/644,911 (Attorney Docket No. 41594-705.101), filed May 9, 2012, the entire content of which is incorporated herein by reference.

### BACKGROUND OF THE INVENTION

1. Field of the Invention

The present invention relates generally to medical apparatus and methods and methods. More particularly, the present invention relates to apparatus and methods for constraining a portion of a blood vessel wall to enhance the response of a stretch receptor in an adjacent region of the wall.

Hypertension is a medical condition characterized by a constant state of elevated blood pressure and can result from a number of underlying factors, including genetics, obesity, diet, and the like. When diagnosed, hypertension is most commonly treated by changes in diet, exercise, and pharmaceutical intervention. More recently, it has been proposed to treat hypertension and related conditions by stimulating or modulating certain stretch receptors, referred to as baroreceptors, in the patient's vasculature. Those receptors are located in the walls of certain blood vessels, such as the carotid arteries and the aortic arch. It has been found that stimulating the baroreceptors and/or the nerves connected to the baroreceptors, can reduce a patient's blood pressure in a clinically effective manner, even when those patients have been resistant to other hypertension treatments.

While a number of these treatments appear to be quite effective in pre-clinical and clinical studies, it would be desirable to provide additional and alternative methods and apparatus for effecting or modulating baroreceptors and other stretch receptors in a clinically effective manner, particularly for the treatment of hypertension and related conditions. At least some of these objectives will be met by the inventions described below.

2. Description of the Background Art

Implantable devices for effecting baroreceptor stimulation are described in US 2008/0033501; US 2001/0077729; US 2011/0178416; 2011/0213408; and 2011/0230953. See also U.S. Pat. No. 6,178,349; U.S. Pat. No. 6,522,926; U.S. Pat. No. 6,850,801; U.S. Pat. No. 7,158,832; U.S. Pat. No. 7,499,747; U.S. Pat. No. 7,499,742; and U.S. Pat. No. 7,502,650.

### SUMMARY OF THE INVENTION

The present invention provides additional and alternative methods and apparatus for stimulating stretch receptors, such as baroreceptors, in a patient's vascular wall. The methods and apparatus are particularly useful for treating hypertension and related conditions, such as congestive heart failure, renal disease, and the like, and may be used as a sole treatment or in combination with other treatments, including pharmaceuticals, lifestyle changes, and other methods and techniques for stimulating or modulating the baroreceptor or other stretch receptor responses.

The present invention differs at least in part from prior methods and apparatus in that an intervention in the vasculature occurs at a region away from the stretch receptor itself. In particular, by constraining portions of a blood vessel wall upstream or downstream of a target stretch receptor, a volumetric or expansion response of the stretch receptor can be amplified or magnified in a manner which will increase the stretch receptor signal which is transmitted by the associated nerves. By “amplification” or “magnification”, it is meant that the signal in response to an increase (or decrease) in pressure within the blood vessel will be greater (or lesser) in response to the intervention than in the absence of the intervention.

In a most straightforward manner, the present invention provides a passive restraint of the blood vessel wall upstream and/or downstream of the target stretch receptor, where the restraint inhibits or prevents expansion and/or contraction of that region of the blood vessel wall. Such constraint, in turn, amplifies or increases the pressure response in an adjacent stretch receptor region of the blood vessel wall which in turn increases the signal generated by the receptor.

In other implementations of the present invention, the restraint could be active. For example, a circumferential or peripheral restraint placed over and/or within the blood vessel can be adjusted in real time after implantation in order to increase, decrease, or provide for feedback control of the stretch receptor signal over time. For example, after implantation, the treating physician can monitor the patient's blood vessel and, if necessary, remotely adjust the degree of constraint provided by the implant using radio frequency or other known techniques for reconfiguring implants. Alternatively, such adjustment could be implemented by minimally invasive surgical techniques.

As a still further alternative, the adjustment of constraint could be performed in response to periodic or real time monitoring of the patient's blood pressure. An implanted or external controller can be configured to receive a signal representative of the patient's blood pressure and to provide a control signal output which can adjust the previously implanted blood vessel wall constraint to provide a desired change in the constraint and resulting change in the amplification of the stretch receptor response.

In a first aspect of the present invention, a method for stimulating a stretch receptor in a vascular wall comprises identifying a region in a patient's vasculature proximate a target stretch receptor, for example, a baroreceptor in a carotid artery wall. A portion of the blood vessel wall upstream and and/or downstream of the target stretch receptor is then constrained in a manner such that the volume displacement during systole and/or diastole is inhibited in the constrained portion and increased in the target region relative to volume displacement in the absence of such constraining

In specific embodiments, the method may further comprise selecting a patient diagnosed with at least one of hypertension, congestive heart failure, renal disease or the like. The stretch receptor will typically comprise a baroreceptor, which is disposed for example in a carotid artery, an aortic arch, or the like. Constraining typically comprises placing a circumferential restraint over an exterior of the blood vessel and/or along or within an internal wall of the blood vessel. The constraint may be elastic, inelastic, or have characteristics intermediate elastic and inelastic. The constraint may be a simple ring or a band which is placed over or within the blood vessel wall, but could be more complex and have regions with different mechanical properties and/or different mechanisms for adjusting the circumferential length or elastic properties. The constraint will typically have an axial length in the range from 0.5 mm to 5 mm, usually from 1 mm to 3 mm, diameter or width in the range from 3 mm to 30 mm, usually from 6 mm to 20 mm. While one constraint will usually be sufficient, in some instances it may be desirable to place two, three, four or even more constraints at locations upstream and/or downstream of the stretch receptor and external and/or internal to the blood vessel wall.

In a second aspect, the present invention provides apparatus for stimulating a stretch receptor. The apparatus comprises a blood vessel wall expansion constraint adapted to be positioned over or within a portion of a blood vessel wall upstream or downstream of a stretch receptor in said blood vessel wall. The expansion constraint typically comprises a circumferential restraint which is adapted to circumscribe the external wall of the blood vessel and/or be secured to an internal wall of the blood vessel immediately upstream or downstream of the target stretch receptor. These specific characteristics of the constraint have been described above in connection with the methods of the present invention.

Suitable materials for the constraint include metals, polymers, meshes, and the like. The materials will be selected to be compatible and suitable for long term implantation with or surrounding the vasculature.

### BRIEF DESCRIPTION OF THE DRAWINGS

FIG. 1 is a schematic illustration of a blood vessel showing an external constraint constructed in accordance with the principles of the present invention located proximate a baroreceptor.

FIG. 2 illustrates a cross section of a blood vessel showing both an external constraint and an internal constraint.

FIGS. 3A and 3B illustrate the effect of the restraints of FIG. 2 in amplifying baroreceptor response.

FIG. 4 illustrates a specific apparatus including a pair of internal constraints joined by a connector.

### DETAILED DESCRIPTION OF THE INVENTION

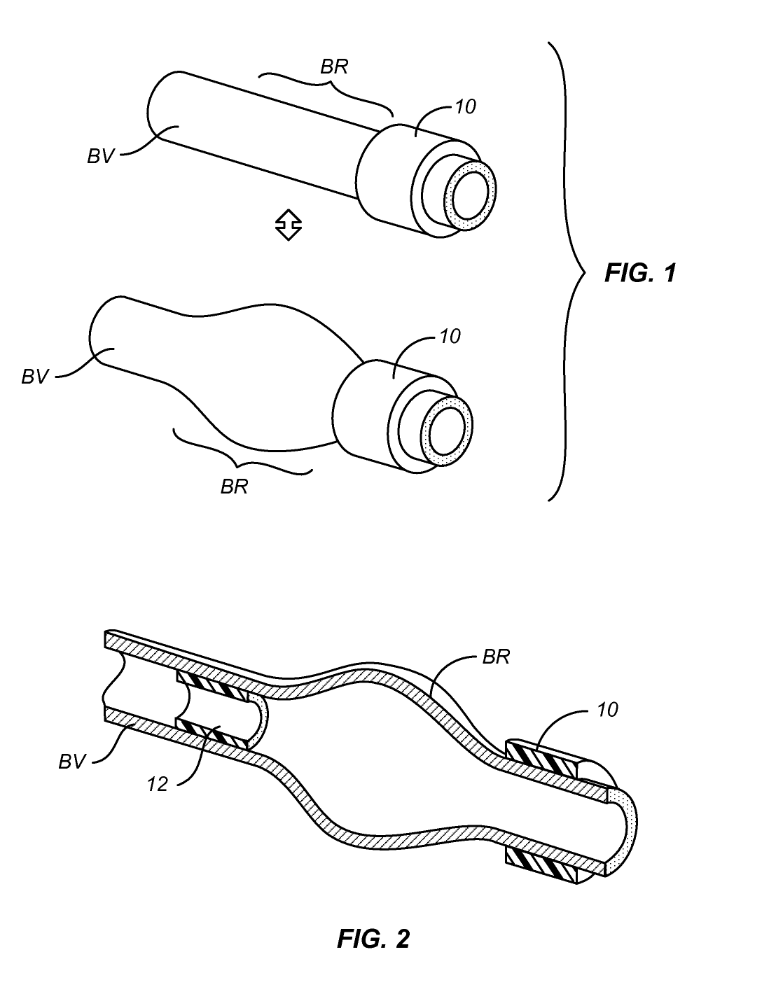

Referring to FIG. 1, a blood vessel BV is illustrated having a baroreceptor region BR and an external restraint or cuff 10 located upstream of the baroreceptor region. As the heart beats, stretching and expansion of the baroreceptor regions BR is enhanced during systole, as shown in the lower portion of FIG. 1. Optionally, in addition to the external cuff 10 shown in FIG. 1, an internal cuff 12 may be provided on the upstream side of the baroreceptor BR, as shown in FIG. 2.

As illustrated, the external cuff 10 and internal cuff 12 are simple cylindrical tubes which are placed on or in the blood vessel BV. The manner of placement will, of course, dictate further structure for the constraints 10 and 12. For example, the external restraint 10 will typically be introduced through open surgical or minimally invasive techniques and will have to be wrapped around the outer wall of the blood vessel. In such instances, usually at least one seam or joint will be needed so that the constraint can be secured after it has been wrapped around the blood vessel. The internal constraint 12, in contrast, will most typically be introduced through an endovascular technique and may be delivered by balloon expansion, self-expansion, or the like. Usually, the internal constraint 12 will have means for embedding the constraint or attaching it to the internal wall of the blood vessel in order to inhibit expansion in response to an increase in pressure within the blood vessel. Techniques for attaching an internal scaffold or constraint to a wall of the blood vessel are known, for example, in connection with vascular grafts which are used, for example, in treatment of aortic abdominal aneurisms. Specific constructions of the external constraint 10 and internal constraint 12 may vary widely within the scope of the present invention so long as they provide for the inhibition or constraint of expansion of the covered wall relative to the unconstrained baroreceptor BR region of the wall.

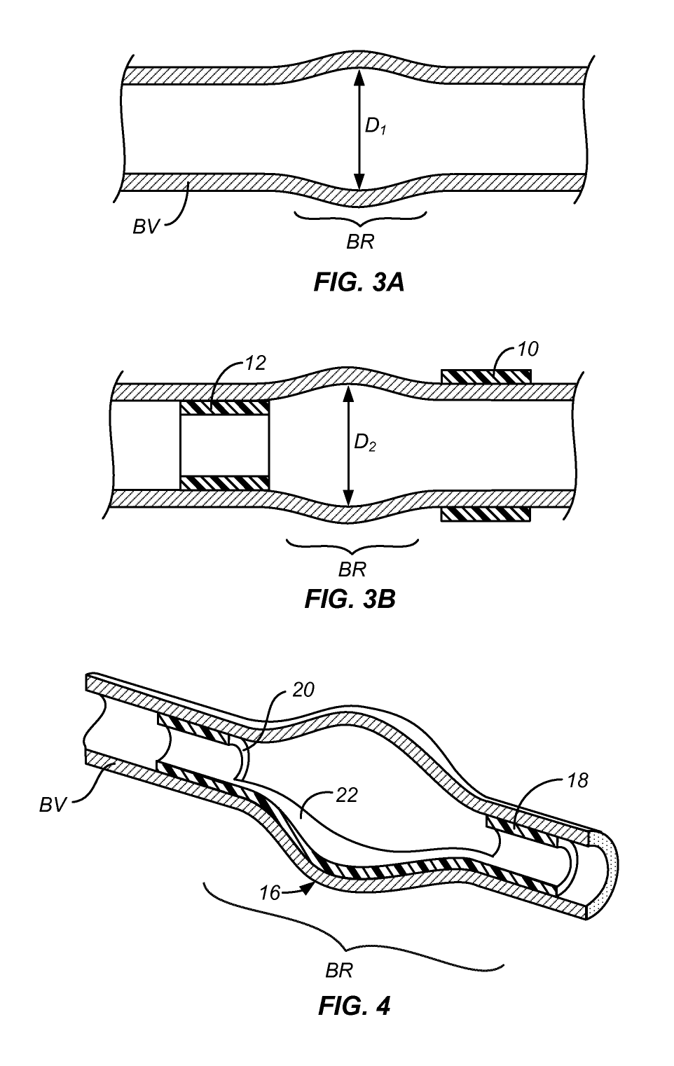

An increase or amplification of the expansion of the baroreceptor BR as a result of the constraint 10 and 12 is illustrated in FIGS. 3A and 3B. FIG. 3A shows a baroreceptor in an unconstrained blood vessel BV or expansion during systole is to a basal diameter D₁. In the presence of constraints 10 and 12, the expansion of the baroreceptor region BR of the blood vessel wall is increased to a greater diameter D₂ as shown in FIG. 3B.

Referring now to FIG. 4, a dual cuff apparatus 16 is illustrated comprising a pair of internal restraints 18 and 20 and a connecting region 22 there between. By coupling or linking the individual restraints 18 and 20, migration of the implanted unit is further inhibited so that they remain properly positioned on either side of the baroreceptor BR region.

What is claimed is:

1. A method for enhancing stimulation of a stretch receptor in a vascular wall, said method comprising:
identifying a region in a patient's vasculature proximate a target stretch receptor; and
constraining a portion of a blood vessel wall upstream or downstream of the target stretch receptor;
wherein volume displacement during systole is inhibited in the contrained portion and increased the target region relative to volume displacement in the absence of such constraining.

2. A method as in claim 1, further comprising selecting a patient diagnosed with at least one of hypertension, congestive heart failure, and renal disease.

3. A method as in claim 1, wherein the stretch receptor comprises a baroreceptor.

4. A method as in claim 3, wherein the stretch receptor is disposed in a carotid artery or an aortic arch.

5. A method as in claim 1, wherein constraining comprises placing a circumferential restraint over an exterior of the blood vessel wall.

6. A method as in claim 5 wherein the constraint has inelastic properties.

7. A method as in claim 5, wherein the constraint has elastic properties.

8. A method as in claim 1, wherein constraining comprises placing a circumferential restraint on an internal wall of the blood vessel.

9. A method as in claim 8, wherein the constraint has inelastic properties.

10. A method as in claim 8, wherein the constraint has elastic properties.

11. A method as in claim 1, wherein the blood vessel wall is constrained over an axial length in the range from 0.5 mm to 5 mm.

12. A method as in claim 1, wherein the blood vessel wall is constrained at at least one location upstream and at at least one location downstream of the target region.

13. A method as in claim 1, wherein the blood vessel wall is constrained at at least one location external to the blood vessel wall and at least one location internal to the blood vessel wall.

14. A method as in claim 5, further comprising adjusting the circumferential length of the restraint after it has been placed.

15. Apparatus for stimulating a stretch receptor, said apparatus comprising:
a blood vessel wall expansion constraint adapted to be positioned over or within a portion of a blood vessel wall upstream or downstream of a stretch receptor in said blood vessel wall.

16. Apparatus as in claim 15, wherein the constraint comprises a circumferential restraint.

17. Apparatus as in claim 16, wherein the circumferential restraint is adapted to be placed over the blood vessel wall.

18. Apparatus as in claim 16, wherein the circumferential restraint is adapted to be placed internally and secured to an interior region of the blood vessel wall.

19. Apparatus as in claim 16, wherein the restraint has inelastic properties.

20. Apparatus as in claim 16, wherein the constraint has elastic properties.

21. Apparatus as in claim 16, wherein the constraint has an axial length in the range from 0.5 mm to 5 mm.

22. Apparatus as in claim 16, wherein the constraint has a circumferential length in the range from 3 mm to 30 mm.

23. Apparatus as in claim 22, wherein the constraint is adjustable.

24. Apparatus as in claim 23, wherein the length is remotely adjustable after implementation.
